(19) United States
(12) Patent Application Publication
(10) Pub. No.: US 2017/0001913 A1
(43) Pub. Date: Jan. 5, 2017

(54) COATING COMPOSITION FOR INHIBITING BUILD-UP OF CARBONACEOUS MATERIAL AND APPARATUS COMPRISING THE COATING AND METHOD

(71) Applicant: General Electric Company, Schenectady, NY (US)

(72) Inventors: Hong ZHOU, Shanghai (CN); Yanfei GU, Shanghai (CN); Shizhong WANG, Shanghai (CN); Wenqing PENG, Shanghai (CN); Wusheng XU, Shanghai (CN); Minghu GUO, Shanghai (CN); Lawrence Bernard KOOL, Clifton Park, NY (US); Zhaohui YANG, Shanghai (CN)

(21) Appl. No.: 15/102,277

(22) PCT Filed: Nov. 24, 2014
(86) PCT No.: PCT/US2014/067089

§ 371 Date: Jun. 7, 2016

(30) Foreign Application Priority Data
Dec. 13, 2013 (CN) 201310686730.7

(51) Int. Cl. C04B 35/50; C04B 35/622; C10G 9/16; C10G 9/20; C01G 25/00; C07C 4/04; B01J 19/00; B01J 19/24; B01J 19/02; C04B 35/634; C10G 75/00

(52) U.S. Cl. CPC C04B 35/50; C04B 35/63416; C04B 35/62222; C10G 9/16; C10G 9/203; C10G 75/00; C07C 4/04; B01J 19/0026; B01J 19/2415; B01J 19/02; C01G 25/006; C04B 2235/3229; C04B 2235/3244; C04B 2235/3215; C04B 2235/3225; B01J 2219/0218; C01P 2002/34

(57) ABSTRACT

A composition useful in methods and apparatuses for inhibiting the build-up of byproduct carbonaceous material includes a perovskite material or a precursor therefor; and a yttrium doped ceria or a precursor therefor.

### BACKGROUND

Embodiments of the present invention relate generally to compositions useful in apparatuses and methods to avoid or reduce the build-up of byproduct carbonaceous material, especially in byproduct carbonaceous material formation environments.

Carbonaceous material is a byproduct of many processes and is usually undesirable. For example, during hydrocarbon cracking processes, the build-up of byproduct carbonaceous materials (i.e. the coke) happens on inner surfaces of apparatus components, for instance, inner radiant tube surfaces of furnace equipment. When the inner radiant tube surfaces become gradually coated with a layer of coke, the radiant tube metal temperature (TMT) rises and the pressure drop through radiant coils increases. In addition, the coke build-up adversely affects the physical characteristics of the apparatus components, e.g., the radiant tubes, by deteriorating mechanical properties such as stress rupture, thermal fatigue, and ductility due to carburization.

Other byproduct coke formation apparatuses and methods, e.g., apparatuses and methods for the steam reforming of methane and for carbonaceous fuel combustion, also have problems caused by the build-up of coke.

A variety of methods have been considered in order to overcome the disadvantages of coke build-up on apparatus components, such as furnace tube inner surfaces. These methods include: metallurgy upgrade to alloys with increased chromium content of the metal substrates used in the apparatuses; and adding additives such as sulfur, dimethyl sulfide (DMS), and dimethyl disulfide (DMDS) or hydrogen sulfide to the feedstock to the apparatuses.

While some of the aforementioned methods have general use in some industries, it is desirable to provide new compositions useful in methods and apparatuses to reduce or eliminate the build-up of carbonaceous material.

### BRIEF DESCRIPTION

In one aspect, the invention relates to a composition comprising: a perovskite material or a precursor therefor; and a yttrium doped ceria or a precursor therefor.

In another aspect, the invention relates to an apparatus having a surface exposable to a byproduct carbonaceous material formation environment and comprising the composition described in the paragraph above.

In yet another aspect, the invention relates to a method comprising: providing the apparatus described in the paragraph above; and exposing the surface to a byproduct carbonaceous material formation environment.

### BRIEF DESCRIPTION OF THE DRAWINGS

These and other features, aspects, and advantages of the present invention will become better understood when the following detailed description is read with reference to the accompanying drawings, wherein:

FIG. 1 illustrates a schematic cross sectional view of a tube of an apparatus according to some embodiments of the invention.

### DETAILED DESCRIPTION

Unless defined otherwise, technical and scientific terms used herein have the same meaning as is commonly understood by one of ordinary skill in the art to which this disclosure belongs. The use of “including”, “comprising” or “having” and variations thereof herein are meant to encompass the items listed thereafter and equivalents thereof as well as additional items.

Approximating language, as used herein throughout the specification and claims, may be applied to modify any quantitative representation that could permissibly vary without resulting in a change in the basic function to which it is related. Accordingly, a value modified by a term or terms, such as “about” is not to be limited to the precise value specified. In some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

In the specification and the claims, the singular forms “a”, “an” and “the” include plural referents unless the context clearly dictates otherwise. Moreover, the suffix “(s)” as used herein is usually intended to include both the singular and the plural of the term that it modifies, thereby including one or more of that term.

As used herein, the term “or” is not meant to be exclusive and refers to at least one of the referenced components (for example, a material) being present and includes instances in which a combination of the referenced components may be present, unless the context clearly dictates otherwise.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances, the modified term may sometimes not be appropriate, capable, or suitable. For example, in some circumstances, an event or capacity can be expected, while in other circumstances, the event or capacity cannot occur. This distinction is captured by the terms “may” and “may be”.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

Embodiments of the present invention relate to compositions useful in methods and apparatuses to avoid or reduce the build-up of byproduct carbonaceous material in byproduct carbonaceous material formation environments.

As used herein the term “carbonaceous material”, “coke”, or any variation thereof refers to but is not limited to carbonaceous solid or liquid, or particulates or macromolecules forming the carbonaceous solid or liquid, which are derived from coal, petroleum, wood, hydrocarbons and other materials containing carbon.

As used herein, the term “byproduct carbonaceous material formation environment” refers to but is not limited to any environments that may yield carbonaceous material as an undesirable byproduct. In some embodiments, the byproduct carbonaceous material formation environment is a petrochemical processing environment. In some embodiments, the byproduct carbonaceous material formation environment is hydrocarbon cracking environment.

In some embodiments, the byproduct carbonaceous material formation environment is a hydrocarbon cracking environment at a temperature in a range from about 700° C. to about 900° C., a weight ratio of steam to hydrocarbon is in a range from about 3:7 to about 7:3, and the hydrocarbon comprises ethane, heptane, liquid petroleum gas, naphtha, gas oil, or any combination thereof.

In some embodiments, the byproduct carbonaceous material formation environment is a hydrocarbon cracking environment at a temperature in a range from about 480° C. to about 600° C., and the hydrocarbon comprises bottoms from atmospheric and vacuum distillation of crude oil and a weight percentage of steam is in a range from about 1 wt % to about 2 wt %.

As used herein the term “hydrocarbon cracking”, “cracking hydrocarbon”, or any variation thereof, refers to but is not limited to processes in which hydrocarbons such as ethane, propane, butane, naphtha, bottoms from atmospheric and vacuum distillation of crude oil, or any combination thereof are cracked in apparatuses to obtain materials with smaller molecules.

As used herein, the term “apparatus” refers to but is not limited to any device that may be exposed to a byproduct carbonaceous material formation environment. In some embodiments, the apparatus includes at least one of a furnace tube, a tube fitting, a reaction vessel, and a radiant tube. The apparatus may be a pyrolysis furnace comprising a firebox through which runs an array of tubing. The array of tubing and corresponding fittings may be several hundred meters in length. The array of tubing may comprise straight or serpentine tubes.

The composition may be in a surface of an apparatus exposed to the byproduct carbonaceous material formation environment, so that the build-up of carbonaceous material on the surface is avoided or reduced.

In some embodiments, the composition includes a combination of the perovskite material and the yttrium doped ceria. In some embodiments, the composition has a combination of the yttrium doped ceria and the precursor for the perovskite material. In some embodiments, the composition comprises a combination of the perovskite material and the precursor for the yttrium doped ceria. In some embodiments, the composition includes a combination of the precursor for the perovskite material and the precursor for the yttrium doped ceria. In some embodiments, the composition comprises a combination of the perovskite material, the precursor for the perovskite material, and the yttrium doped ceria. In some embodiments, the composition includes a combination of the perovskite material, the yttrium doped ceria and the precursor for the yttrium doped ceria. In some embodiments, the composition has a combination of the perovskite material, the precursor for the perovskite material, the yttrium doped ceria and the precursor for the yttrium doped ceria.

The amount of the yttrium doped ceria or the precursor therefor and the perovskite material or the precursor therefor in the composition may vary depending on the specific materials being used and the working conditions of the composition, as long as the composition inhibits the build-up the byproduct carbonaceous material. In some embodiments, a weight ratio of the yttrium doped ceria to the perovskite material is in a range from about 0.1:99.9 to about 99.9:0.1, or from about 1:9 to about 9:1, or more particularly from about 1.5:100 to about 9:10.

As used herein the term “perovskite material” or any variation thereof refers to but is not limited to any material having an ABO₃ perovskite structure and being of formula AₐB_{b}O_{3-δ}, wherein 0.9<a≦1.2; 0.9<b≦1.2; −0.5<δ<0.5; A comprises a first element and optionally a second element, the first element is selected from calcium (Ca), strontium (Sr), barium (Ba), lithium (Li), sodium (Na), potassium (K), rubidium (Rb) and any combination thereof, the second element is selected from yttrium (Y), bismuth (Bi), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu) and any combination thereof; and B is selected from silver (Ag), gold (Au), cadmium (Cd), cerium (Ce), cobalt (Co), chromium (Cr), copper (Cu), dysprosium (Dy), erbium (Er), europium (Eu), ferrum (Fe), gallium (Ga), gadolinium (Gd), hafnium (Hf), holmium (Ho), indium (In), iridium (Ir), lanthanum (La), lutetium (Lu), manganese (Mn), molybdenum (Mo), niobium (Nb), neodymium (Nd), nickel (Ni), osmium (Os), palladium (Pd), promethium (Pm), praseodymium (Pr), platinum (Pt), rhenium (Re), rhodium (Rh), ruthenium (Ru), antimony (Sb), scandium (Sc), samarium (Sm), tin (Sn), tantalum (Ta), terbium (Tb), technetium (Tc), titanium (Ti), thulium (Tm), vanadium (V), tungsten (W), yttrium (Y), ytterbium (Yb), zinc (Zn), zirconium (Zr), and any combination thereof.

In some embodiments, the perovskite material may be of formula n(AₐB_{b}O_{3-δ}), in which n=2, 3, 4, 8, and etc., and the formula AₐB_{b}O_{3-δ} is the simplified form thereof.

In some embodiments, in the ABO₃ perovskite structure, A cations are surrounded by twelve anions in cubo-octahedral coordination, B cations are surrounded by six anions in octahedral coordination and oxygen anions are coordinated by two B cations and four A cations. In some embodiments, the ABO₃ perovskite structure is built from corner-sharing BO₆ octahedra. In some embodiments, the ABO₃ perovskite structure includes distorted derivatives. The distortions may be due to rotation or tilting of regular, rigid octahedra or due to the presence of distorted BO₆ octahedra. In some embodiments, the ABO₃ perovskite structure is cubic. In some embodiments, the ABO₃ perovskite structure is hexagonal.

The first element may be a single element or a combination of elements, selected from calcium (Ca), strontium (Sr), barium (Ba), lithium (Li), sodium (Na), potassium (K), and rubidium (Rb). In some embodiments, A only comprises the first element.

In some embodiments, A comprises a combination of the first element and the second element. The second element may be a single element or a combination of elements selected from yttrium (Y), bismuth (Bi), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

Likewise, B may be a single element or a combination of elements selected from silver (Ag), gold (Au), cadmium (Cd), cerium (Ce), cobalt (Co), chromium (Cr), copper (Cu), dysprosium (Dy), erbium (Er), europium (Eu), ferrum (Fe), gallium (Ga), gadolinium (Gd), hafnium (Hf), holmium (Ho), indium (In), iridium (Ir), lanthanum (La), lutetium (Lu), manganese (Mn), molybdenum (Mo), niobium (Nb), neodymium (Nd), nickel (Ni), osmium (Os), palladium (Pd), promethium (Pm), praseodymium (Pr), platinum (Pt), rhenium (Re), rhodium (Rh), ruthenium (Ru), antimony (Sb), scandium (Sc), samarium (Sm), tin (Sn), tantalum (Ta), terbium (Tb), technetium (Tc), titanium (Ti), thulium (Tm), vanadium (V), tungsten (W), yttrium (Y), ytterbium (Yb), zinc (Zn), and zirconium (Zr).

In some embodiments, the perovskite material comprises SrCeO₃, SrZr_{0.3}Ce_{0.7}O₃, BaMnO₃, BaCeO₃, BaZr_{0.3}Ce_{0.7}O₃, BaZr_{0.3}Ce_{0.5}Y_{0.2}O₃, BaZr_{0.1}Ce_{0.7}Y_{0.2}O₃, BaZrO₃, BaZr_{0.7}Ce_{0.3}O₃, BaCe_{0.5}Zr_{0.5}O₃, BaCe_{0.9}Y_{0.1}O₃, BaCe_{0.85}Y_{0.15}O₃, BaCe_{0.8}Y_{0.2}O₃, or any combination thereof. For example, for SrCeO₃, A is Sr, a=1, B is Ce, b=1, and δ=0. For SrZr_{0.3}Ce_{0.7}O₃, A is Sr, a=1, B is a combination of Zr and Ce, b=1, and δ=0. For BaMnO₃, A is Ba, a=1, B is Mn, b=1, and δ=0. For BaCeO₃, A is Ba, a=1, B is Ce, b=1, and δ=0. For BaZr_{0.3}Ce_{0.7}O₃, A is Ba, a=1, B is a combination of Zr and Ce, b=1, and δ=0. For BaZr_{0.3}Ce_{0.5}Y_{0.2}O₃, A is Ba, a=1, B is a combination of Zr, Ce and Y, b=1, and δ=0.

In some embodiments, the perovskite material comprises La_{0.1}Ba_{0.9}Ce_{0.7}Zr_{0.2}Y_{0.1}O₃, Ce_{0.1}Ba_{0.9}Ce_{0.7}Zr_{0.2}Y_{0.1}O_{3.05}, Ce_{0.5}Ba_{0.5}Ce_{0.7}Zr_{0.2}Y_{0.1}O_{3.45}, Y_{0.1}Ba_{0.9}Ce_{0.7}Zr_{0.2}Y_{0.1}O₃, Y_{0.5}Ba_{0.5}Ce_{0.7}Zr_{0.2}Y_{0.1}O_{3.2}, Bi_{0.1}Ba_{0.9}Ce_{0.7}Zr_{0.2}Y_{0.1}O₃, Bi_{0.5}Ba_{0.5}Ce_{0.7}Zr_{0.2}Y_{0.1}O_{3.2}, Pr_{0.1}Ba_{0.9}Ce_{0.7}Zr_{0.2}Y_{0.1}O₃, Pr_{0.5}Ba_{0.5}Ce_{0.7}Zr_{0.2}Y_{0.1}O_{3.2}, or any combination thereof. For La_{0.1}Ba_{0.9}Ce_{0.7}Zr_{0.2}Y_{0.1}O₃, A is a combination of Ba and La, the first element is La, the second element is Ba, a=1, B is a combination of Ce, Zr and Y, b=1, and, δ=0. For Ce_{0.1}Ba_{0.9}Ce_{0.7}Zr_{0.2}Y_{0.1}O_{3.05} and Ce_{0.5}Ba_{0.5}Ce_{0.7}Zr_{0.2}Y_{0.1}O_{3.45}, A is a combination of Ce and Ba, the first element is Ce, the second element is Ba, a=1, B is a combination of Ce, Zr and Y, b=1, and, δ=−0.05 and −0.45, respectively. For Y_{0.1}Ba_{0.9}Ce_{0.7}Zr_{0.2}Y_{0.1}O₃ and Y_{0.5}Ba_{0.5}Ce_{0.7}Zr_{0.2}Y_{0.1}O_{3.2}, A is a combination of Y and Ba, the first element is Y, the second element is Ba, a=1, B is a combination of Ce, Zr and Y, b=1, and, δ=0 and −0.2, respectively. For Bi_{0.1}Ba_{0.9}Ce_{0.7}Zr_{0.2}Y_{0.1}O₃ and Bi_{0.5}Ba_{0.5}Ce_{0.7}Zr_{0.2}Y_{0.1}O_{3.2}, A is a combination of Bi and Ba, the first element is Bi, the second element is Ba, a=1, B is a combination of Ce, Zr and Y, b=1, and, δ=0 and −0.2, respectively. Similarly, for Pr_{0.1}Ba_{0.9}Ce_{0.7}Zr_{0.2}Y_{0.1}O₃ and Pr_{0.5}Ba_{0.5}Ce_{0.7}Zr_{0.2}Y_{0.1}O_{3.2}, A is a combination of Pr and Ba, the first element is Pr, the second element is Ba, a=1, B is a combination of Ce, Zr and Y, b=1, and, δ=0 and −0.2, respectively.

The precursor for the perovskite material may be any material that leads to the formation of the perovskite material. In some embodiments, the precursor for the perovskite material comprises a combination of a carbonate of A and an oxide of B, or a precursor for the carbonate of A or the oxide of B. In some embodiments, the precursor for the perovskite material comprises a combination of an oxacid salt of A and B, or a precursor therefor. In some embodiments, the precursor for the perovskite material comprises a combination of barium carbonate, zirconia, and ceria.

In some embodiments, the yttrium doped ceria comprises YₓCe₁₋ₓO_{2≠α}, wherein 0<x<1, and 0<α<0.5. In some embodiments, the yttrium doped ceria comprises Y_{0.1}Ce_{0.9}O_{1.95}.

The precursor for the yttrium doped ceria may be any precursor that leads to the formation of the yttrium doped ceria. In some embodiments, the precursor comprises a combination of cerium oxide and yttrium oxide. In some embodiments, the precursor for the yttrium doped ceria comprises a combination of oxacid salts of yttrium and cerium.

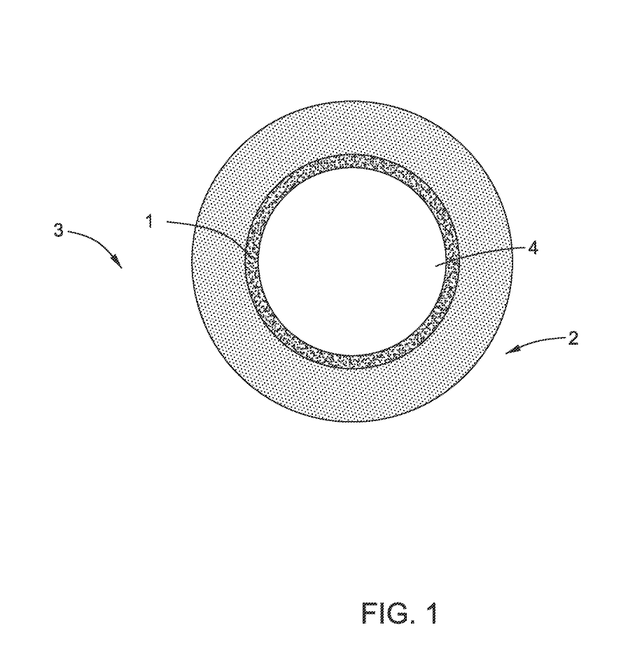

In some embodiments, the surface of the apparatus exposed to the byproduct carbonaceous material formation environment comprises a coating of the composition. In some embodiments, as is shown in FIG. 1, the surface 1 comprises an inner surface of a tube 2 of an apparatus 3, and the byproduct carbonaceous material formation environment 4 is inside the tube 2.

The composition may be coated to the apparatus using different methods, for example, air plasma spray, slurry coating, sol-gel coating, solution coating, or any combination thereof.

In some embodiments, the composition is slurry coated to the apparatus. The amount of the composition in the slurry may vary as long as a continuous, strong, and anticoking coating is formed, depending on the specific materials being used and the working conditions of the coating. In some embodiments, a weight ratio of the yttrium doped ceria to the perovskite material in the slurry is in a range from about 0.1:99.9 to about 99.9:0.1, or from about 1:9 to about 9:1, or more particularly from about 1.5:100 to about 9:10.

The slurry may further comprise an organic binder, an inorganic binder, a wetting agent, a solvent or any combination thereof to enhance the slurry wetting ability, tune the slurry viscosity and get a good green coating strength. When the organic binder, the inorganic binder, the wetting agent, the solvent, or any combination thereof is added in the slurry, a total weight percentage of the composition in the slurry may be from about 10% to about 90%, or from about 15% to about 70%, or more particularly from about 30% to about 55%.

In some embodiments, the slurry may be applied to the surface of the apparatus by different techniques, such as sponging, painting, centrifuging, spraying, filling and draining, dipping, or any combination thereof. In some embodiments, the slurry is applied by dipping, i.e., dipping the part of the apparatus to be coated in the slurry. In some embodiments, the slurry is applied by filling and draining, i.e., filling the slurry in the tube of the apparatus to be coated and draining out the slurry afterwards by, e.g., gravity.

In some embodiments, after the slurry is applied to the apparatus, the coated apparatus is sintered to obtain a coating with a good strength at a high temperature. As used herein the term “sintering” or any variations thereof refers to, but is not limited to, a method of heating the material in a sintering furnace or other heater facility. In some embodiments, the sintering temperature is in a range from about 850° C. to about 1700° C. In some embodiments, the sintering temperature is at about 1000° C.

In sintering, the perovskite material or the precursor therefor may or may not chemically react with the yttrium doped ceria or the precursor therefor. Thus, the coating may comprise a combination or a reaction product of the perovskite material or the precursor therefor and the yttrium doped ceria or the precursor therefor. In some embodiments, the perovskite material comprises yttrium and/or cerium from the yttrium doped ceria or the precursor therefor.

As can be seen from following examples, the coating comprising the composition has a surprisingly high strength.

### EXAMPLES

The following examples are included to provide additional guidance to those of ordinary skill in the art in practicing embodiments of the claimed invention. These examples do not limit the embodiments of the present invention as defined in the appended claims.

### Example 1

### BaZr_{0.3}Ce_{0.7}O₃ powder preparation

The BaZr_{0.3}Ce_{0.7}O₃ powder was prepared by solid-state reaction method. Stoichiometric amounts of high-purity barium carbonate, zirconium oxide, and cerium oxide powders (all from sinopharm chemical reagent Co., Ltd. (SCRC), Shanghai, China) were mixed in ethanol and ball-milled for 16 hours. The resultant mixtures were then dried and calcined at 1450° C. in air for 6 hours to form the BaZr_{0.3}Ce_{0.7}O₃ powder. The calcined powder was mixed with alcohol and was ball milled for 16 hours. After the alcohol was dried, the fine BaZr_{0.3}Ce_{0.7}O₃ powder (d₅₀=1.5 micron) was prepared.

### Example 2

### Slurry Preparation

BaZr_{0.3}Ce_{0.7}O₃ fine powder prepared in example 1 and different amounts (details are shown in table 1 below) of CeO₂ sol (20 wt % suspension in H₂O, Alfa Aesar #12730, from Alfa Aesar Company, Ward Hill, Mass., USA), Y₂O₃ (AR, sinopharm chemical reagent Co., Ltd. (SCRC), Shanghai, China), glycerol (AR, sinopharm chemical reagent Co., Ltd. (SCRC), Shanghai, China), poly(vinyl alcohol) (PVA, molecular weight: 88,000-97,000) 10 wt % aqueous solution, and water were respectively added into plastic jars mounted on speed mixer machines. After mixing for 3 minutes with the rotation speed of 3000 revolutions per minute (RPM), respective slurries were prepared.

TABLE 1

| | slurry | slurry | slurry | slurry | slurry | slurry | slurry | slurry | slurry | slurry | slurry |
|---|---|---|---|---|---|---|---|---|---|---|---|
| | 1 | 2 | 3 | 4 | 5 | 6 | 7 | 8 | 9 | 10 | 11 |
| BaZr_{0.3}Ce_{0.7}O₃ | 8.87 | 6.28 | 6.28 | 6.65 | 6.95 | 7.35 | 5.78 | 10 | 5.90 | 6.63 | 7.87 |
| powder (g) | | | | | | | | | | | |
| CeO₂ sol | 11.92 | 11.92 | 11.92 | 11.92 | 11.92 | 11.92 | 11.92 | 7 | 11.92 | 0 | 0 |
| (20 wt % | | | | | | | | | | | |
| suspension) (g) | | | | | | | | | | | |
| Y₂O₃ (g) | 0.35 | 0.16 | 0.165 | 0.348 | 0.553 | 0.783 | 0 | 0 | 0 | 0 | 0.25 |
| glycerol (g) | 1.09 | 1.09 | 1.09 | 1.09 | 1.09 | 1.09 | 1.09 | 1.38 | 1.09 | 1.17 | 1.09 |
| PVA (10 wt % | 3.21 | 3.21 | 3.21 | 3.21 | 3.21 | 3.21 | 3.21 | 4.09 | 3.21 | 1.3 | 3.21 |
| aqueous | | | | | | | | | | | |
| solution) (g) | | | | | | | | | | | |
| H₂O (g) | 0 | 0 | 0 | 0 | 0 | 0 | 0 | 0 | 0 | 3.9 | 0 |
| Molar ratio of | 35 | 42 | 42 | 42 | 42 | 42 | 43 | 20 | 42 | 0 | 30 |
| CeO₂ & Y₂O₃/ | | | | | | | | | | | |
| (CeO₂ & Y₂O₃ + | | | | | | | | | | | |
| BaZr_{0.3}Ce_{0.7}O₃) | | | | | | | | | | | |
| (%) | | | | | | | | | | | |
| Molar ratio | 1/9 | 5/95 | 5/95 | 1/9 | 15/85 | 2/8 | 0 | 0 | 0 | 0 | 0 |
| Y₂O₃/CeO₂ | | | | | | | | | | | |

### Example 3

### Applying the Slurries on Coupons

A plurality of coupons made from stainless steel each with the dimension of 10×30×1 mm³ were used as substrates. The substrates were cleaned carefully as follows: ultrasonic agitation in acetone and ethanol for 5 minutes respectively to remove organic contaminants, ultrasonic agitation in HCl (3.3 wt %) aqueous solution for 5 minutes to remove metal oxides, ultrasonically rinsing in deionized water, and dried using compressed air.

Cleaned coupons were dipped into the slurries prepared in EXAMPLE 2 and was then lifted out with the speed of 70 mm/min. The coated coupons were dried in air for 2 hours at 80° C. and were then put into a furnace for sintering at 1000° C. for 3 hours in vacuum before being cooled to the room temperature. The increasing and decreasing rates of temperature in the furnace were 1° C./min or 6° C./min.

It was observed that materials of the coatings using slurries 1-9 are bonded together at different degrees but materials of the coatings using slurries 10-11 are barely bonded with each other and easily come off from the coupons, suggesting the strengths of coatings using slurries 10-11 are weak after sintering at 1000° C. The results indicate organic binders decomposed during sintering and yttrium oxide alone is not a good binder.

### Example 4

### XRD Analysis

X-ray diffraction (XRD) analyses were conducted to examine the coatings on the coupons. Y_{0.1}Ce_{0.9}O_{1.95} and BaZr_{0.3}Ce_{0.7}O₃ were detected in the XRD analyses of the coupons coated using slurries 1-6. It suggests that a reaction between Y₂O₃ and CeO₂ happened and yttrium entered the crystal structure of CeO₂ to form yttrium doped ceria.

Regarding the coupons coated using slurries 7-9, there were no shiftings of BaZr_{0.3}Ce_{0.7}O₃ peaks with CeO₂ percentage increasing with regard to the coupon coated using slurry 10, which indicates that no significant reactions took place between CeO₂ and BaZr_{0.3}Ce_{0.7}O₃.

According to XRD quantification of the coating using the slurry 11, yttrium entered the BaZr_{0.3}Ce_{0.7}O₃ crystal structure.

### Example 5

### SEM Analysis

The coatings on the coupons were studied by scanning electron microscope (SEM) analysis. BaZr_{0.3}Ce_{0.7}O₃ powders were bonded better in the coatings of the coupons coated using slurries 1-6 than in the coatings of the coupons coated using slurries 7-9, in which were better than in the coating of the coupon coated using the slurry 10. The densities of the coatings using slurries 1-6 were higher than those of the coatings of the coupons coated using slurries 7-9 which were higher than that of the coating of the coupon coated using the slurry 10. Therefore, the coating strength gets higher with the addition of CeO₂ and even higher with further addition of Y₂O₃ in the slurry, although Y₂O₃ alone is not a good binder.

### Example 6

### Pencil Test

Pencil hardness test was employed to measure the hardness of the coatings for obtaining the levels of the cohesive strengths of the coatings. Coatings of slurries 1-6 had hardnesses of H while coatings of slurries 7 and 9 had hardnesses of HB, the coating of slurry 8 had a hardness of 5B, and the coating of slurry 11 had a hardness of less than 5B. The pencil test shows that Y₂O₃ alone does not improve the coating strength, a combination of Y₂O₃ and CeO₂ surprisingly and significantly enhances the coating hardness from less than 5B, 5B or HB to H and hence significantly improve the coating strength, which is probably because of the formation of the yttrium doped ceria in sintering.

### Example 7

### Tape Testing

Tape testing standard method, which is based on ASTM D3359, was employed to test the adherent strength of coatings on the coated coupon. Damages of coatings on coupons coated using slurries 1-6 are smaller than those of coatings on coupons coated using slurries 7-10, thereby the coating adhesion strengths of coupons coated using slurries 1-6 were stronger than those of coupons coated using slurries 7-10.

This tape testing result was well consistent with the coating surface morphology by SEM analysis in example 5 and the results of the pencil test in example 6.

### Example 8

### Hydrocarbon Cracking

Coupons coated using slurries 1-10 in example 3 were placed on alumina sample holders at the constant temperature region of a lab scale hydrocarbon-cracking furnace. The furnace door was then closed. Argon gas was fed in the furnace at the flow rate of 100 standard cubic centimeters per minute (sccm). The cracking furnace was heated to about 870° C. with the ramping rate of about 20° C./min. A vaporizer was heated to about 350° C. within about 30 minutes.

When the temperature of the cracking furnace reached about 870° C. and the temperature of the vaporizer reached about 350° C., water was pumped using a piston pump into the vaporizer with the flow rate of about 1.59 ml/min. Argon gas feeding was stopped. After about 5 minutes, heptane was pumped using a piston pump into the vaporizer with the flow rate of about 2.26 ml/min to be vaporized and mixed with the steam in the vaporizer in a 1:1 weight ratio. The temperature of the cracking furnace was maintained at desired temperature, e.g., about 870±5° C. for about 1.5 hours before stopping the pumping of the heptane and water. The residence time of the heptane and steam in the cracking furnace was about 1.5 seconds, unless otherwise specified. Argon gas was fed again at the flow rate of about 100 sccm before the cracking furnace and the vaporizer were shut down. When the cracking furnace cooled down, argon gas feed was stopped and the furnace door was opened to take out the sample holders.

No coke was observed on any of the coatings of the coupons but cokes were found on uncoated parts of all the coupons, which indicate the coatings are anticoking.

While only certain features of embodiments of the present invention have been illustrated and described herein, many modifications and changes will occur to those skilled in the art. It is, therefore, to be understood that the appended claims are intended to cover all such modifications and changes as fall within the true spirit of embodiments of the present invention.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A composition comprising:
a perovskite material or a precursor therefor; and
a yttrium doped ceria or a precursor therefor.

2. The composition of claim 1, wherein the perovskite material is of formula AₐB_{b}O_{3-δ}, wherein
0.9<a≦1.2;
0.9<b≦1.2;
−0.5≦δ≦0.5;
A comprises a first element and optionally a second element, the first element is selected from calcium (Ca), strontium (Sr), barium (Ba), lithium (Li), sodium (Na), potassium (K), rubidium (Rh) and any combination thereof, the second element is selected from yttrium (Y), bismuth (Bi), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu) and any combination thereof; and
B is selected from silver (Ag), gold (Au), cadmium (Cd), cerium (Ce), cobalt (Co), chromium (Cr), copper (Cu), dysprosium (Dy), erbium (Er), europium (Eu), ferrum (Fe), gallium (Ga), gadolinium (Gd), hafnium (Hf), holmium (Ho), indium (In), iridium (Ir), lanthanum (La), lutetium (Lu), manganese (Mn), molybdenum (Mo), niobium (Nb), neodymium (Nd), nickel (Ni), osmium (Os), palladium (Pd), promethium (Pm), praseodymium (Pr), platinum (Pt), rhenium (Re), rhodium (Rh), ruthenium (Ru), antimony (Sb), scandium (Sc), samarium (Sm), tin (Sn), tantalum (Ta), terbium (Tb), technetium (Tc), titanium (Ti), thulium (Tm), vanadium (V), tungsten (W), yttrium (Y), ytterbium (Yb), zinc (Zn), zirconium (Zr), and any combination thereof.

3. The composition of claim 1, wherein the yttrium doped ceria comprises YₓCe₁₋ₓO_{2±α}, wherein 0<x<1, and 0<α<0.5.

4. The composition of claim 1, wherein the precursor for yttrium doped ceria comprises a mixture of yttria and ceria.

5. The composition of claim 1, wherein the perovskite material comprises BaZr_{0.3}Ce_{0.7}O₃.

6. The composition of claim 1, wherein a weight ratio of the perovskite material to the yttrium doped ceria is in a range from about 0.1:99.9 to about 99.9:0.1.

7. An apparatus having a surface exposable to a byproduct carbonaceous material formation environment, the surface comprising a composition comprising: a perovskite material or a precursor therefor; and a yttrium doped ceria or a precursor therefor.

8. The apparatus of claim 7, wherein the yttrium doped ceria comprises YₓCe₁₋ₓO_{2±α}, wherein 0<x<1, and 0<α<0.5.

9. The apparatus of claim 7, wherein the perovskite material comprises BaZr_{0.3}Ce_{0.7}O₃.

10. The apparatus of claim 7, wherein a weight ratio of the perovskite material to the yttrium doped ceria is in a range from about 0.1:99.9 to about 99.9:0.1.

11. The apparatus of claim 7, wherein the surface comprises a coating of the composition.

12. The apparatus of claim 7, wherein the composition is slurry coated and sintered and the precursor for yttrium doped ceria comprises a mixture of ceria and yttria.

13. The apparatus of claim 7, wherein the surface comprises an inner surface of a tube and comprises the perovskite material and the yttrium doped ceria.

14. A method, comprising:
providing an apparatus having a surface comprising a composition comprising: a perovskite material or a precursor therefor; and a yttrium doped ceria or a precursor therefor; and
exposing the surface to a byproduct carbonaceous material formation environment.

15. The method of claim 14, wherein the perovskite material comprises BaZr_{0.3}Ce_{0.7}O₃.

16. The method of claim 14, wherein the yttrium doped ceria comprises Y_{0.1}Ce_{0.9}O_{1.95}.

17. The method of claim 14, wherein the byproduct carbonaceous material formation environment is a hydrocarbon cracking environment and the hydrocarbon comprises ethane, heptane, liquid petroleum gas, naphtha, gas oil, bottoms from atmospheric and vacuum distillation of crude oil, or any combination thereof.

18. The method of claim 14, wherein the surface comprises an inner surface.

19. The method of claim 14, wherein the surface comprises an inner surface of a tube.

20. The method of claim 14, wherein the surface comprises a coating of the composition.
